(19) United States
(12) Patent Application Publication
(10) Pub. No.: US 2026/0207235 A1
(43) Pub. Date: Jul. 23, 2026

(54) BONE SCREWS

(71) Applicant: GLW, Inc., Englewood Cliffs, NJ (US)

(72) Inventors: Axel Cremer, Fahrenkrug (DE); Richard Garret Mauldin, Erie, CO (US); Torben Bröhan, Englewood Cliffs, NJ (US); Henrik Nuesse, Englewood Cliffs, NJ (US); Vadim Gurevich, Englewood Cliffs, NJ (US)

(73) Assignee: GLW, Inc., Englewood Cliffs, NJ (US)

(21) Appl. No.: 19/324,731

(22) Filed: Sep. 10, 2025

Related U.S. Application Data
(60) Provisional application No. 63/692,935, filed on Sep. 10, 2024.
(60) Provisional application No. 63/692,929, filed on Sep. 10, 2024.
(60) Provisional application No. 63/692,941, filed on Sep. 10, 2024.

(51) Int. Cl. A61B 17/86

(52) U.S. Cl. CPC A61B 17/864; A61B 17/8605; A61B 17/8635

(57) ABSTRACT

Various example bone screws are described.

### FIELD

The disclosure relates generally to medical devices. More particularly, the disclosure relates to bone screws useful in orthopedic procedures.

### BACKGROUND

Bone screws are widely used in orthopedic procedures for the fixation of bone fractures and stabilization of bone structures.

While efforts have been made to address limitations of currently available bone screws, a need remains for bone screws with improved characteristics, such as cutting performance, ease of removal, adaptability for demands of different procedures, anatomies, and care provider preferences, and desirable impacts on inventory management and procedure preparation.

### BRIEF SUMMARY OF SELECTED EXAMPLES

Various example bone screws are described.

An example bone screw comprises a body having a body proximal end, a body distal end, and a body length extending between the body proximal end and the body distal end; a proximal portion having a proximal portion proximal end, a proximal portion external thread, and a proximal portion length extending from the proximal portion proximal end to the body proximal end, the proximal portion length being less than the body length; a distal tip having a cutting surface and extending from the body distal end; and a head extension disposed on the proximal portion and having a head extension internal thread threaded with the proximal portion external thread form.

An example device is a cannulated bone screw. The cannulated bone screw includes a screw head, a screw tip, a screw body, and a helical thread form. The screw head is disposed at a proximal end of the cannulated bone screw. The screw tip is disposed at a distal end of the cannulated bone screw. The screw body is disposed between and extending from both the screw head and the screw tip. The screw body defines an outer wall and an inner wall opposite the outer wall. The inner wall has a first inner wall portion and a second inner wall portion. The first inner wall portion defines a first lumen. The second inner wall portion defines a second lumen. The second lumen is in fluid communication with the first lumen. A first circumference of the first inner wall portion is greater than a second circumference of the second inner wall portion.

An example method is a method for installing and removing a cannulated bone screw in a subject. The method includes inserting a cannulated bone screw into a bone of the subject. The cannulated bone screw comprises a screw head, a screw tip, a screw body, and a helical thread form. The screw head is disposed at a proximal end of the cannulated bone screw. The screw tip is disposed at a distal end of the cannulated bone screw. The screw body is disposed between and extending from both the screw head and the screw tip. The screw body defines an outer wall and an inner wall opposite the outer wall. The inner wall has a first inner wall portion and a second inner wall portion. The first inner wall portion defines a first lumen. The second inner wall portion defines a second lumen. The second lumen is in fluid communication with the first lumen. A first circumference of the first inner wall portion is greater than a second circumference of the second inner wall portion. The method further comprises rotating the cannulated bone screw to engage the helical thread form with the bone.

### BRIEF DESCRIPTION OF FIGURES

FIG. 1 is a perspective view of an example cannulated bone screw.

FIG. 2 is a lengthwise sectional view of a prior art cannulated bone screw.

FIG. 3 is a lengthwise sectional view of the example cannulated bone screw illustrated in FIG. 1.

FIG. 4 is a partial side view of a proximal end of a prior art beveled bone screw.

FIG. 5 is a partial side view of a proximal end of an example bone screw having a radiused bevel and domed terminal surface.

FIG. 6 is a partial perspective view of the example bone screw illustrated in FIG. 5.

FIG. 7A is perspective view of an example headless head extension for a bone screw.

FIG. 7B is sectional view of the example headless head extension illustrated in FIG. 7A.

FIG. 8A is a schematic illustration of the installation of the example headless head extension illustrated in FIG. 7A onto a preexisting bone screw.

FIG. 8B is a schematic illustration of the example headless head extension illustrated in FIG. 7A installed on a preexisting bone screw.

FIG. 9 is a perspective view of an example headed head extension.

FIG. 10A is a schematic illustration of the installation of the example headed head extension illustrated in FIG. 9 onto a preexisting bone screw.

FIG. 10B is a schematic illustration of the example headed head extension illustrated in FIG. 9 installed on a preexisting bone screw.

FIG. 11 is a perspective of another example headless head extension.

FIG. 12A is a schematic illustration of the installation of the example headless head extension illustrated in FIG. 11 onto a preexisting bone screw.

FIG. 12B is a schematic illustration of the example headless head extension illustrated in FIG. 11 installed on a preexisting bone screw.

FIG. 13 is a schematic illustration of the installation of a headless head extension onto a preexisting bone screw.

FIG. 14A is an illustration of a headless head extension.

FIG. 14B is an illustration of a beveled head extension.

FIG. 14C is an illustration of a headless head extension.

FIG. 14D is an illustration of a headed head extension.

FIG. 15 is a perspective view of another example headless head extension.

FIG. 16 is a partial sectional view of a bone screw with the example headless head extension illustrated in FIG. 15 installed.

FIG. 17 is a perspective view of another example headless head extension.

FIG. 18A is a partial perspective view of a bone screw with the example headless head extension illustrated in FIG. 17 installed.

FIG. 18B is a partial perspective view of the bone screw illustrated in FIG. 18A.

FIG. 19 is a perspective view of another example headless head extension.

FIG. 20A is a partial perspective view of a bone screw with the example headless head extension illustrated in FIG. 19 installed.

FIG. 20B is a partial perspective view of the bone screw illustrated in FIG. 20A.

FIG. 21 is a schematic illustration of the installation of the example headless head extension illustrated in FIG. 19 onto a preexisting bone screw.

FIG. 22 is a schematic illustration of an example kit including a bone screw body and multiple head extensions.

FIG. 23 is schematic illustration of the installation of an example clip on extension onto a preexisting bone screw.

FIG. 24A is a partial sectional view of a bone screw with an example clip on head extension.

FIG. 24B is a partial sectional view of a bone screw with another example clip on head extension.

FIG. 24C is a partial sectional view of a bone screw with another example clip on head extension.

### DETAILED DESCRIPTION OF SELECTED EXAMPLES

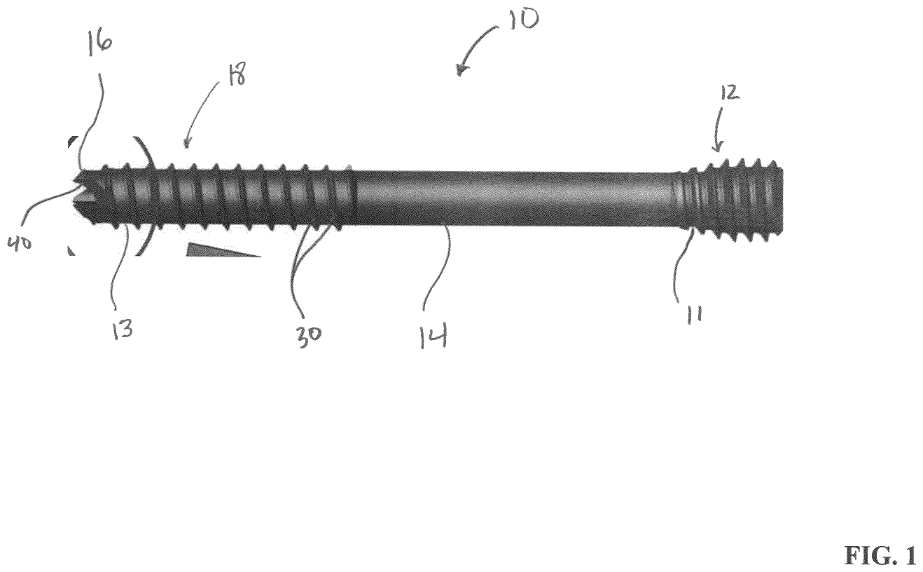

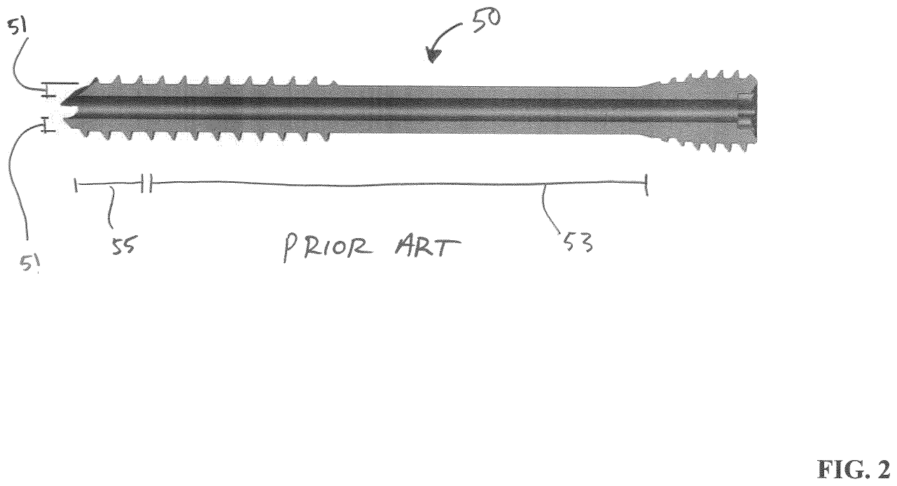

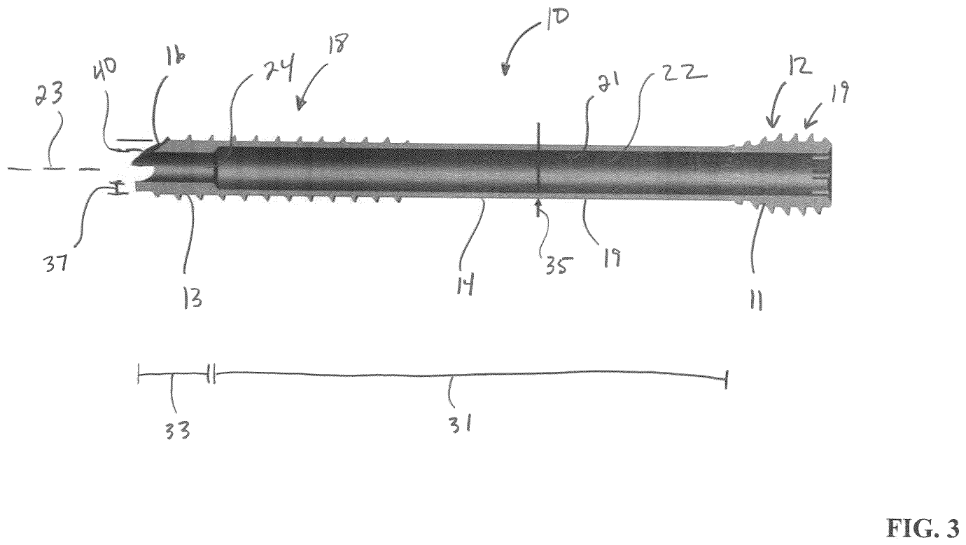

Each of FIGS. 1 and 3 illustrates an example cannulated bone screw 10. FIG. 2 illustrates a prior art cannulated bone screw 50.

The cannulated bone screw 10 includes a cannulated bone screw head 12, a cannulated bone screw proximal end 11, a cannulated bone screw distal end 13, and a cannulated bone screw body 14. The cannulated bone screw 10 defines a cannulated bone screw proximal end 11 and a cannulated bone screw distal end 13. For this example, proximal is considered the direction that is closer to a user installing the cannulated bone screw 10 and distal is considered the direction that is distant from the user installing the cannulated bone screw 10. The cannulated bone screw head 12 is located at the cannulated bone screw proximal end 11. A cannulated bone screw tip 16 is located at the cannulated bone screw distal end 13. The cannulated bone screw body 14 is disposed between the cannulated bone screw head 12 and the cannulated bone screw tip 16. Further, the cannulated bone screw body 14 extends from both the cannulated bone screw head 12 and the cannulated bone screw tip 16 toward the other.

The cannulated bone screw proximal end 11 is the end of the cannulated bone screw 10 positioned closer to the user during the installation process. The cannulated bone screw proximal end 11 serves as the point where the user interfaces with the screw to apply the necessary torque for insertion into the bone. The cannulated bone screw proximal end 11 also houses part of the first lumen 22, which extends into and through the cannulated bone screw head 12. This configuration allows the screw to accommodate various medical instruments and/or guide wires. The cannulated bone screw 10 is configured for rotation, enabling the user to insert or remove the screw depending on the direction of rotation.

The cannulated bone screw distal end 13 is located farther from the user during installation and includes the cannulated bone screw tip 16. The cannulated bone screw distal end 13 is configured to engage with the bone as the cannulated bone screw 10 is inserted. The cannulated bone screw tip 16 defines a second lumen 24, which is in fluid communication with the first lumen 22 extending from the proximal end 11. The cannulated bone screw tip 16 also includes a tip helical thread form 18, which extends from the cannulated bone screw body 14 and is configured to aid in securing the bone screw during installation. The tip helical thread form 18 further allows the screw to anchor into the bone by engaging with the bone surface. Additionally, the cannulated bone screw tip 16 is configured with a plurality of taps 40, which includes cutting edges designed to assist in driving the screw into the bone. In some embodiments, rotating the screw in the opposite direction allows the removal of the cannulated bone screw 10 from the subject.

The cannulated bone screw head 12 is located at the proximal end 11 of the cannulated bone screw 10. The cannulated bone screw head 12 is configured to receive a driver, allowing a user to interface with the cannulated bone screw 10. This interface enables the user to rotate the cannulated bone screw head 12, driving the cannulated bone screw 10 into bone. In some embodiments, the cannulated bone screw head 12 is rotated in a first direction to advance the cannulated bone screw 10 into the bone. Additionally, the cannulated bone screw head 12 is rotated in a second, opposite direction to remove the cannulated bone screw 10 from the bone. The configuration of the cannulated bone screw head 12 serves as an interface for a driver. The driver interface enables both installation and removal processes.

The cannulated bone screw tip 16 of the cannulated bone screw 10 is located at the distal end 13. The cannulated bone screw tip 16 is configured to engage with the bone during installation of the bone screw. The cannulated bone screw tip 16 is tapered. Tapering allows the bone screw to gradually penetrate the bone. The cannulated bone screw tip 16 taper includes a plurality of tapered angles to facilitate insertion at various stages of engagement with the bone. The cannulated bone screw tip 16 also includes a plurality of taps 40. The plurality of taps 40 are configured to be cutting features configured to aid in the installation process. The plurality of taps 40 create threads in the bone as the screw is rotated. Even further, the plurality of taps 40 is configured to remove small amounts of bone material to allow the tip helical thread form 18 to grip securely into the bone surface. This interaction between the plurality of taps 40 and the bone improves the stability of the screw within the bone. In some embodiments, the plurality of taps 40 aids in removing the screw when rotated in the opposite direction, aiding in removing the screw from the bone.

The cannulated bone screw body 14 is disposed between the cannulated bone screw head 12 and the cannulated bone screw tip 16. The cannulated bone screw body 14 extends from both the cannulated bone screw head 12 and the cannulated bone screw tip 16 and defines an outer wall 19 and an inner wall 21. The inner wall 21 defines the first lumen 22 and the second lumen 24 that are in fluid communication with each other. The first lumen 22 has a larger circumference than the second lumen 24, allowing for the accommodation of various instruments or fluids during surgical procedures. The cannulated bone screw body 14 defines a central axis 23 extending along the general center of both the first lumen 22 and the second lumen 24. The outer wall 19 of the cannulated bone screw body 14 includes a helical thread form, which aids in the engagement and anchoring of the screw to the bone as it is rotated into place. This configuration allows the cannulated bone screw 10 to effectively secure to the bone while allowing fluid communication within its lumens for medical use.

As best illustrated in FIG. 3, the first lumen 22 extends along a first portion 31 of the bone screw 10 that includes the bone screw body 14. The second lumen 24 extends along a second portion 33 of the bone screw that includes the distal end 13. In the first portion 31, the outer wall 19 and inner wall 21 define a first wall thickness 35. In the second portion 33, the outer wall 19 and inner wall 21 define a second wall thickness 37 that is greater than the first wall thickness 35. In the illustrated example, the second wall thickness 37 is at least twice the first wall thickness 35. Other relative wall thicknesses can be used. For example, in some examples, the second wall thickness is greater than twice the first wall thickness. In some examples, the second wall thickness is at least two and a half times the first wall thickness. In some examples, the second wall thickness is at least three times the first wall thickness. In some examples, the second wall thickness is at least four times the first wall thickness. In some examples, the second wall thickness is at least five times the first wall thickness. In some examples, the second wall thickness is at least ten times the first wall thickness.

As illustrated in FIG. 2, a cannulated bone screw 50 according to the prior art includes a continuous wall thickness 51 that extends along first 53 and second 55 portions of the cannulated bone screw 50.

The tip helical thread form 18 of the cannulated bone screw 10 extends along at least the cannulated bone screw tip 16 and continues onto the outer wall of the cannulated bone screw body 14. The tip helical thread form 18 is configured to engage with the bone when the screw is rotated. When the tip helical thread form 18 engages with the bone, it provides an anchoring force that secures the cannulated bone screw 10 within the bone. The tip helical thread form 18 includes a first thread pattern. The first thread pattern is defined by a plurality of ridges. In some embodiments, a second thread pattern is present. In the second thread pattern, the ridges is thicker than those of the first pattern. Additionally, the tip helical thread form 18 includes a plurality of rakes 30 positioned to aid in the removal of the screw when rotated in the opposite direction.

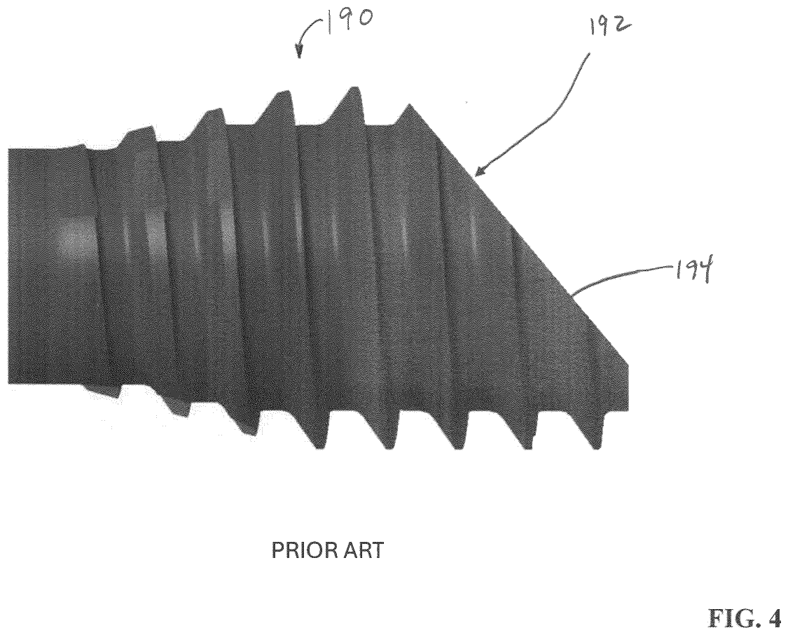

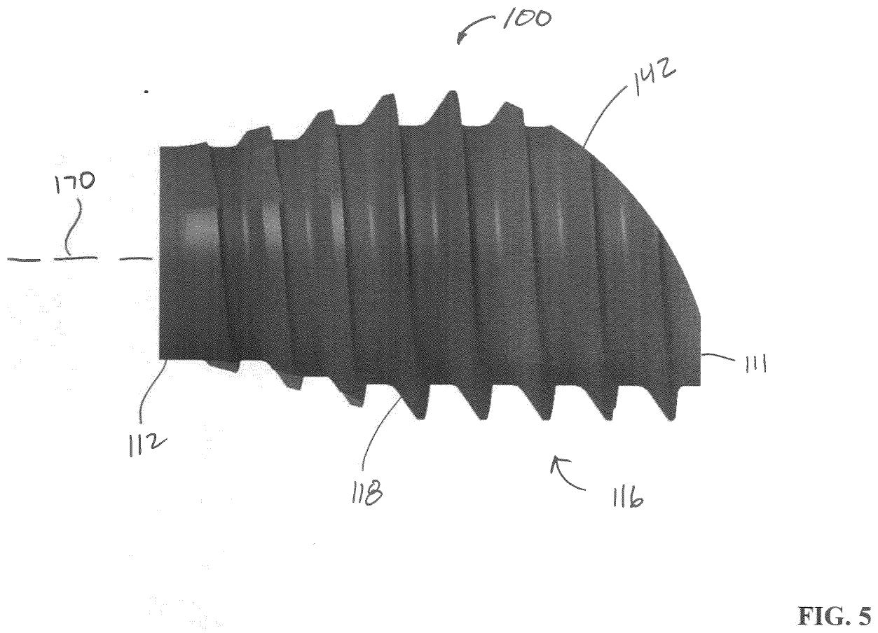

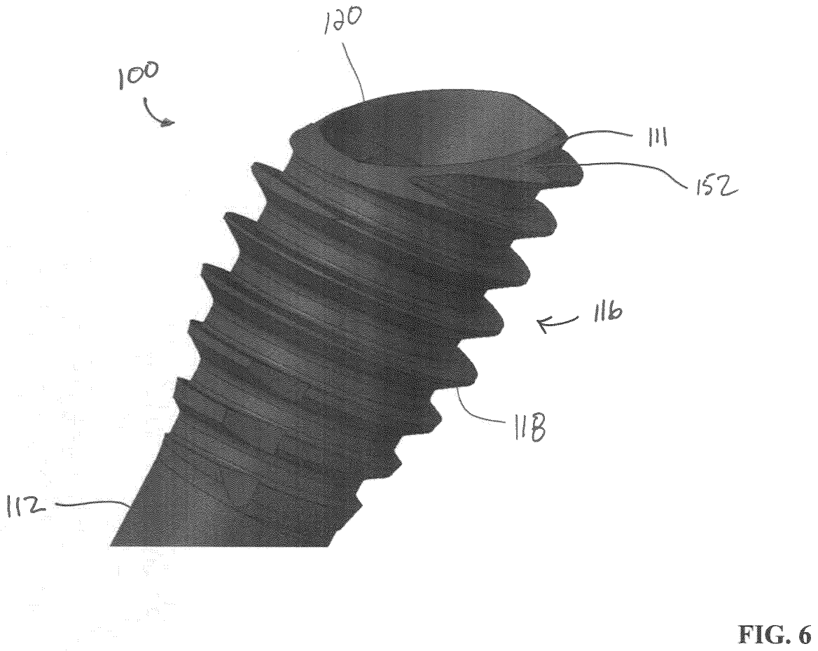

Each of FIGS. 5 and 6 illustrates the proximal end of another example bone screw 100. Bone screw 100 has a radiused bevel and domed proximal surface, as described in detail below. The remainder of bone screw 100 can be in accordance with example bone screw 10 described above. FIG. 4 illustrates the proximal end of a prior art bone screw 190 having a flat bevel 192 and planar proximal surface 194.

Bone screw 100 includes a bone screw body 112, a bone screw tip (not illustrated), and a bone screw head 116. The bone screw body 112 is disposed between the bone screw tip and the bone screw head 116. The bone screw body 112 extends longitudinally from the bone screw head 116 at the bone screw proximal end 111 to the bone screw tip 114 at the bone screw distal end (not illustrated).

The bone screw body 112 may be cylindrical in shape and may align with the central screw axis 170. The bone screw proximal end 111 is the end closest to the installer during the procedure, allowing for the application of a torque force during installation. The bone screw distal end (not illustrated), located further from the installer, includes the bone screw tip, which is configured to penetrate bone. The bone screw head 116 is disposed at the bone screw proximal end 111.

The bone screw tip is located at the bone screw distal end of the bone screw 110. The bone screw tip serves as the primary point of contact with the bone during installation. The bone screw tip is configured to taper toward the bone screw distal end, allowing for easier insertion into the bone by gradually engaging with the bone's surface as the screw is rotated.

The bone screw tip may include a helical thread, which extends from the bone screw body 112 toward the bone screw distal end. This helical thread may be configured to aid in anchoring the screw within the bone by cutting into the bone material as the screw rotates. In addition, the bone screw tip may include cutting elements, such as taps or rakes, that assist in the initial penetration of the bone and the formation of threads, which enhance the stability of the screw once fully inserted. The bone screw tip may also be configured to allow for reverse rotation, facilitating easy removal when necessary.

The bone screw body 112 is positioned between the bone screw tip and the bone screw head 116, extending longitudinally from the bone screw proximal end 111 to the bone screw distal end. The bone screw body 112 serves as the main structural component. The bone screw body 112 generally defines the central screw axis 170, which extends longitudinally along the length of the screw 100. In some embodiments, the bone screw body 112 may be cannulated, allowing for the insertion of guide wires or other surgical instruments through the body. In other embodiments, the bone screw body 112 may be solid, providing additional strength for certain applications. Other configurations of the bone screw body 112 may include variations in thickness or the presence of internal threads to accommodate specific medical requirements.

The bone screw head 116 is located at the bone screw proximal end 111. The bone screw head 116 serves as the interface for the installer to apply torque during installation. The bone screw head 116 is connected to the bone screw body 112. The bone screw head includes a driver interface 120. The bone screw head 116 is configured to receive a driver into the driver interface 120. This allows the installer to rotate the screw for proper placement in the bone. The driver interface 120 may be shaped in a variety of configurations.

The bone screw head 116 defines a bone screw head plane, which is angularly displaced from the central screw axis 170 by a bevel angle. This bevel angle may vary depending on factors such as the purpose of the screw, the area of bone targeted for implantation, or the type of bone material. The bone screw head plane is intersected by the central screw axis 170. The bevel angle enhances the anchoring capabilities of the screw by optimizing the contact surface with the bone.

The bone screw head 116 being beveled provides multiple advantages. A beveled head allows for greater bone contact during insertion, which improves the stability of the screw without wasting bone matter. The bevel also ensures that the depth of the screw's implantation correlates with the stability of the anchor in the bone, as deeper implantation provides increased bone engagement. The beveled screw head 116 permits the operator to interface with the screw more easily, even when the alignment of the driver interface with the driver is not perfect. This is particularly useful when operators encounter difficulty in precisely aligning the driver with the screw head. A curved driver interface may allow for a greater margin of error during installation, making it easier for the operator to engage the screw in challenging surgical conditions.

The depth of the screw implantation directly affects its stability, especially in dense bone regions such as cortical bone. If the screw is implanted too shallow, it may not anchor properly. Conversely, deeper implantation provides a more secure hold in the bone, increasing the ability of the screw to resist forces and maintain its position over time.

In the illustrated embodiment, a helical thread form 118 is present on the bone screw head 116. The head helical thread form 118 may be generally parallel to and radially spaced from the central screw axis 170, helping to maintain proper alignment during insertion. In other embodiments, however, the helical thread form 118 on the screw head may not be parallel or radially spaced from the central screw axis 170.

The helical thread form 118 may be partially broken away on the bone screw head 16, allowing for a lower profile and minimizing potential interference with surrounding tissue. In other embodiments, the helical thread form 118 may extend completely up to the head, maximizing engagement with the bone for procedures requiring greater anchoring strength. Some embodiments may omit the helical thread form 118 on the bone screw head 116 altogether.

Furthermore, the helical thread form 118 may include a variety of thread patterns, with different widths and thicknesses tailored for specific bone types or surgical needs. These variations in the thread layout help to optimize the screw's interaction with bone tissue. The helical thread form 118 may also incorporate rakes, which assist in the removal of the screw by creating a more controlled exit path during reverse rotation.

As best illustrated in FIG. 4, prior art beveled bone screws include a flat bevel 192 and flat planar proximal surface 194 on the bone screw proximal end 111, providing the interface between the bone screw 110 and the driver.

The flat bevel 192 and flat planar proximal surface 194 provide minimal tolerance for installation errors and requires precise alignment with the driver. It must be flush with the driver interface for correct usage. Due to the nature of the flat planar proximal surface 194, which lacks curvature, there is only a single correct installation angle. Any deviation from this angle may result in the screw head protruding from the bone, which could cause soft tissue irritation around the site of implantation.

Even when the straight beveled screw head of bone screw 190 is installed at the proper angle, it is highly likely that part of the material will still protrude from the bone, particularly because the bone is typically rounded while the screw head has a flat bevel 192 and flat planar proximal surface 194. This mismatch between the screw head and the bone surface may lead to protrusions, which could cause discomfort or require additional surgical adjustments.

Furthermore, the flat bevel 192 and flat planar proximal surface 194 may move slightly relative to the bone during the installation process, as the screw is rotated into place, which emphasizes the need for consistent alignment to avoid complications. Its flat surface and single-angle installation requirement present a greater challenge compared to other head configurations like the radiused or domed variants.

As best illustrated in FIGS. 5 and 6, the radiused beveled screw head 116 is located at the bone screw proximal end 111. This screw head includes a radiused surface 142, which is curved and has a radiused perimeter. The radiused surface 142 may be shaped to conform to a curved profile that increases the interaction between the screw and the bone during insertion. The radiused surface 142 and its radiused perimeter generally lie outside the bone screw head plane, but both may rise at multiple points into the bone screw head plane, depending on the bevel angle and the radius profile.

One advantage of the radiused beveled screw head 116 is its greater margin of error compared to the straight beveled screw head. The curvature of the radiused surface 142 may allow for more flexibility during installation, permitting slight variations in the angle between the driver and the driver interface 120. This greater tolerance may prove beneficial in scenarios where precision alignment is difficult, as the radiused beveled screw head 116 may still engage with the driver even when there is minor misalignment in at least one vector.

However, the radiused beveled screw head 116 may still present challenges. If the installation angle is not optimal, portions of the screw material may protrude from the bone. Even when installed correctly, the curved nature of the radiused surface 142, combined with the naturally rounded shape of bone, increases the likelihood that some material will extend beyond the bone surface, potentially causing soft tissue irritation.

The radiused perimeter further supports the interaction between the screw head and the bone by providing a continuous curved outline, which may improve the distribution of force during insertion. While the radiused surface 142 offers increased tolerance, it is still required that the screw be installed within a specific range of angles to avoid protrusion issues and ensure proper placement within the bone structure.

As best illustrated in FIG. 6, a radiused surface 142 can provide a domed surface 152 positioned at the bone screw proximal end 111, serving as the interface for applying torque to the bone screw 110 during the installation process. This screw head includes a domed surface 152, which is frustospherical in shape. The domed surface 152 and the domed perimeter generally extend outside the bone screw head plane, but in certain configurations, a single point of the surface may rise into the plane. This configuration allows for greater adaptability when installing the screw into a bone, especially in rounded bone surfaces.

The domed beveled screw head 116 provides the greatest margin of error among similar configurations, such as the straight beveled screw head or a radiused beveled screw head that is not a domed beveled screw head. Its frustospherical shape allows the installer to vary the angle of the driver without compromising the screw's interaction with the driver interface 120. This facilitates ease of use in surgical conditions, where precision alignment of the driver may be difficult. Furthermore, the domed shape may prevent excessive protrusion of screw material from the bone, especially in cases where a bone has a rounded surface that matches the contour of the domed surface 152.

Additionally, the domed beveled screw head limits the angles at which the screw material extends from the bone, maintaining a lower profile after installation. This minimizes the risk of soft tissue irritation. The domed configuration is beneficial when the screw is installed at the proper angle, ensuring that the material does not extend from the bone surface due to its rounded configuration, which conforms more closely to the natural bone shape.

The domed beveled screw head 116 is positioned at the bone screw proximal end 11, interfacing with the bone screw body 112 to facilitate torque application during screw installation. The domed surface 152 is frustospherical in shape and is defined by a combination of geometric elements, including a rotation axis, a curve axis, and a radii point. The rotation axis is generally parallel and axially spaced apart from the bone screw head plane. This axis serves as the reference for the overall orientation of the screw head, enabling rotation of the bone screw 100 during insertion into the bone.

Head extensions are provided as improvements to bone screws. Head extensions are configured to be disposed on the bone screw head of an existing bone screw, such as a bone screw according to an embodiment described herein. The extensions are specifically configured to extend the existing bone screw head. In some embodiments, this will increase the interface between the bone and the screw for greater support.

At least three types of head extensions are presented. This includes a beveled head extension (245), headless head extensions (230, 240, 260, 265, 270), and headed head extensions (235, 250). However, the specific head extensions mentioned are not to be seen as limiting, as other configurations are included. Additionally, the proximal portion 216 may come prefabricated with one of the mentioned extensions. These prefabricated configurations may streamline the surgical procedure by providing the installer with a bone screw that is ready for specific applications.

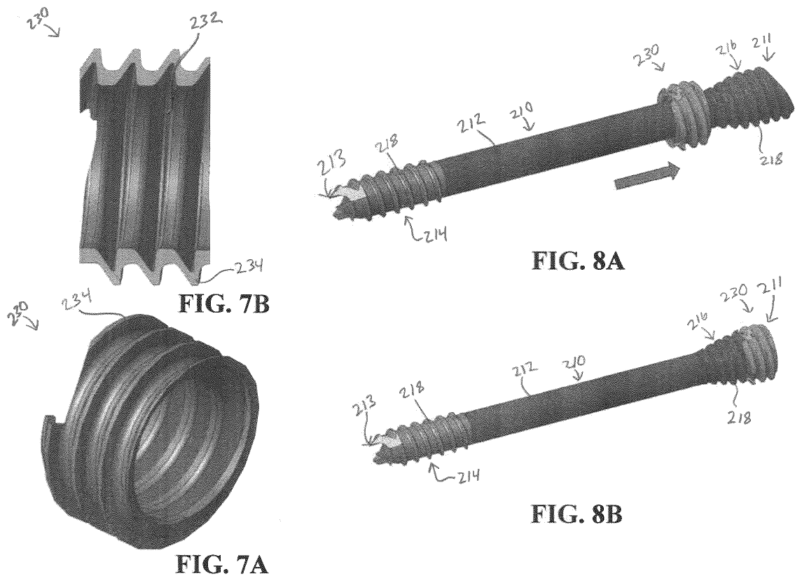

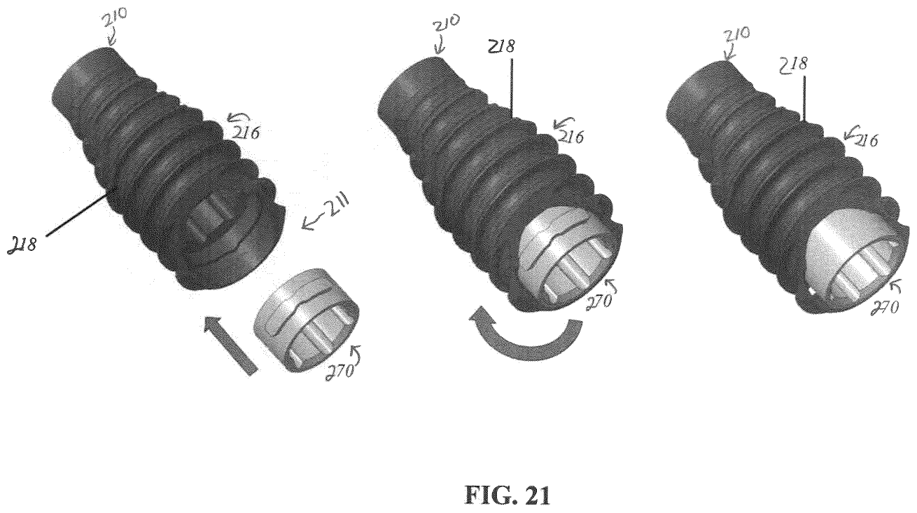

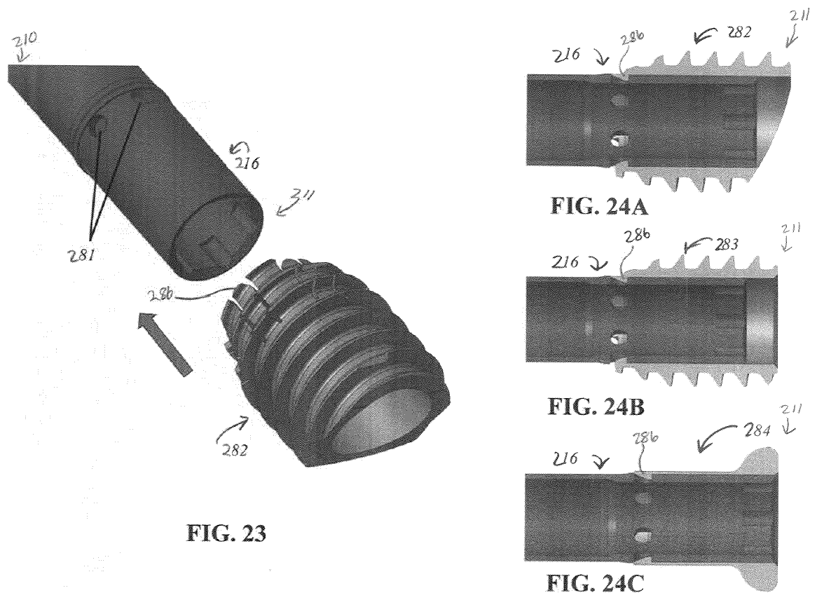

Each of FIGS. 7A, 7B, 8A, 8B, 9, 10A, 10B, 11, 12A, 12B, 13, 14A, 14B, 14C, 14D, 15, 16, 17, 18A, 18B, 19, 20A, 20B, 21, 22, 23, 24A, 24B, and 24C illustrates an example head extension according to the present disclosure. In these illustrations, bone screws 210 include a body 212, a proximal end 211, a distal end 213, a distal tip 214, a proximal portion 216, and one or more thread forms 218.

Each of FIGS. 7A, 7B, 8A, and 8B illustrates a first headless head extension 230. The first headless head extension 230 is configured to be loaded distally onto the bone screw 210. The head extension 230 has a diameter that is greater than a diameter of the body 212. The first headless head extension 230 includes an internal thread 232 that is configured to traverse past a helical thread form 218 positioned on either the distal end 213 or the proximal end 211 of the bone screw 210. The internal thread 232 ensures that the first headless head extension 230 can be securely positioned along the bone screw 210. In the illustrated embodiment, the helical thread form 218 on the proximal portion 216 is tapered. The first headless head extension 230 may use this tapering by using the tension generated as the first headless head extension 230 is threaded proximally, allowing the tapering to function as a stopping point. The first headless head extension 230 also includes an external thread form 234, which may act as an extension of the helical thread form 218 disposed on the proximal portion 216, when the first headless head extension 230 is in its final position. This continuity between the external thread form 234 of the first headless head extension 230 and the proximal portion 216 increases the anchoring ability of the bone screw 210 while implemented in the bone.

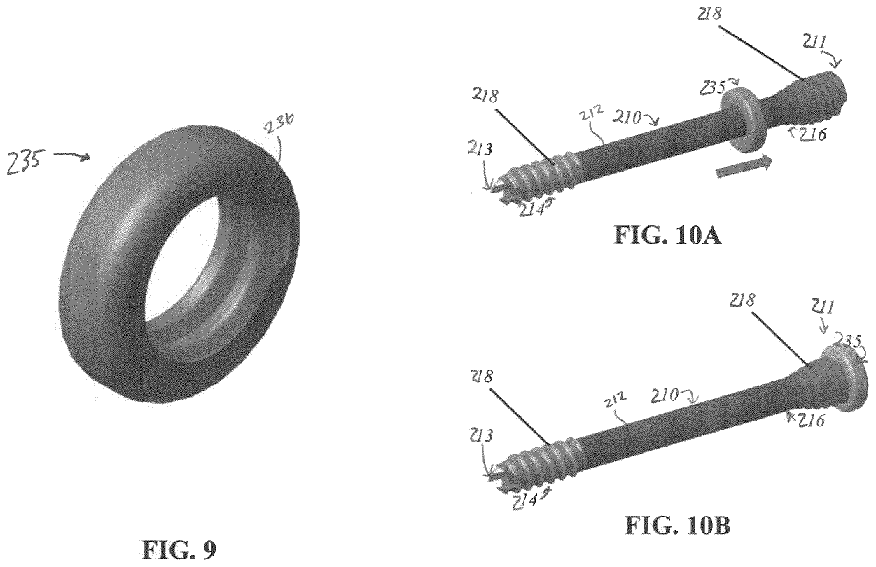

Each of FIGS. 9, 10A, and 10B illustrates a first headed head extension 235. The first headed head extension 235 is configured to be loaded distally onto the bone screw 210. The first headed head extension 235 includes an internal thread 236. The internal thread 236 is configured to traverse past a helical thread form 218, which can be positioned either on the distal end 213 or proximal end 211 of the bone screw 210. In the illustrated embodiment, the helical thread form 218 on the proximal portion 216 is tapered. The first headed head extension 235 may use the tension created by the tapering to serve as a stopping point.

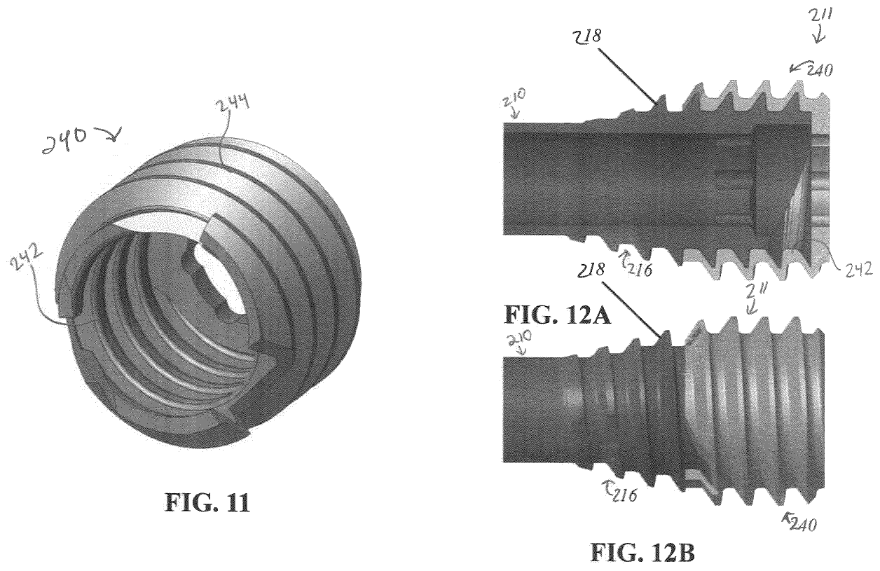

Each of FIGS. 11, 12A, and 12B illustrates a second headless head extension 240. The second headless head extension 240 is configured to be loaded proximally onto the bone screw 210. The second headless head extension 240 includes an internal thread 242. The internal thread 242 is configured to traverse past a portion of the helical thread form 218 positioned on the bone screw proximal end 211. Upon rotation of the second headless head extension 240, it may abut the proximal portion 216, which may serve as a stopping point. This abutment further ensures that the second headless head extension 240 is properly aligned and prevents further rotation beyond the intended position.

The second headless head extension 240 also includes an external thread 244. When the second headless head extension 240 reaches the stopping point, the external thread 244 of the second headless head extension 240 is continuous with the helical thread form 218 on the proximal portion 216. This continuity enhances the stability of the bone screw 210 and ensures seamless engagement between the second headless head extension 240 and the bone screw 210.

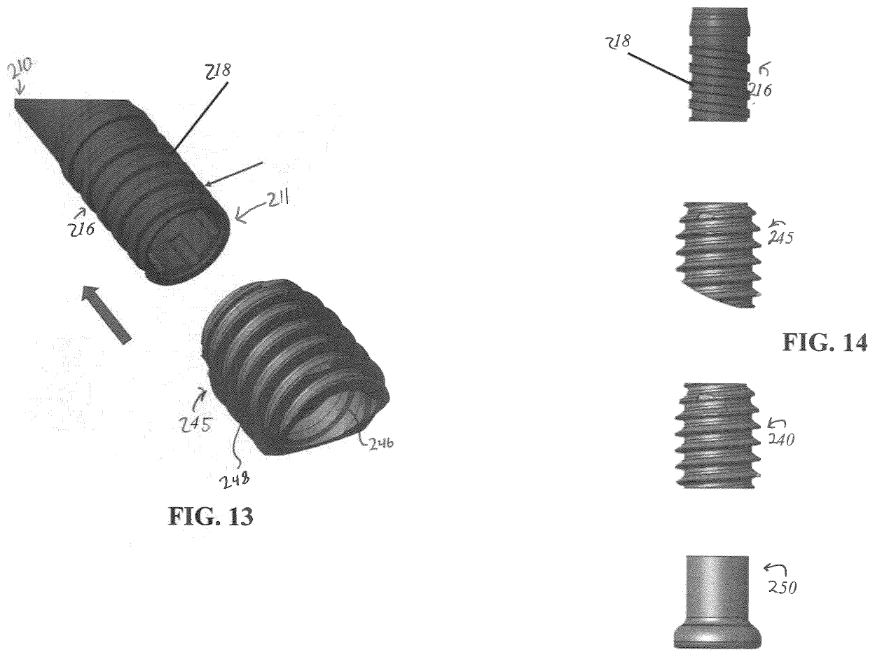

Each of FIGS. 13 and 14B illustrates a first beveled head extension 245. The first beveled head extension 245 is configured to be loaded proximally onto the bone screw 210. The first beveled head extension 245 includes an internal thread 246, which is configured to traverse past a portion of the head helical thread form 218 positioned on the proximal end 211 of the bone screw 210. The internal thread 246 ensures a secure and proper fit as the first beveled head extension 245 is rotated onto the proximal portion 216.

After rotation of the first beveled head extension 245 onto the proximal portion 216, the first beveled head extension 245 may abut the proximal portion 216. This abutment acts as a stopping point, preventing further rotation and ensuring proper positioning of the first beveled head extension 245 relative to the bone screw 210.

Additionally, the first beveled head extension 245 includes an external thread 248. Upon reaching the stopping point, the external thread 248 of the first beveled head extension 245 is continuous with the helical thread form 218 on the proximal portion 216. This continuity enhances the overall stability and engagement of the bone screw 210 within the bone.

FIG. 14D illustrates the second headed head extension 250 as part of a bone screw assembly. The second headed head extension 250 is configured to be loaded proximally onto the bone screw 210. During installation, the second headed head extension 250 is threaded onto the bone screw 210 from the proximal end 211, interacting with the proximal portion 216. The second headed head extension 250 includes an internal thread, not illustrated. The internal thread is configured to traverse past a portion of the helical thread form 218 that is positioned on the proximal end 211 of the bone screw 210. As the second headed head extension 250 is rotated onto the bone screw 210, it moves along the helical thread form 218 until it abuts the proximal portion 216. This abutment acts as a stopping point, ensuring the second headed head extension 250 is correctly positioned relative to the proximal portion 216. Once the second headed head extension 250 reaches this stopping point, it is securely seated, preventing further rotation.

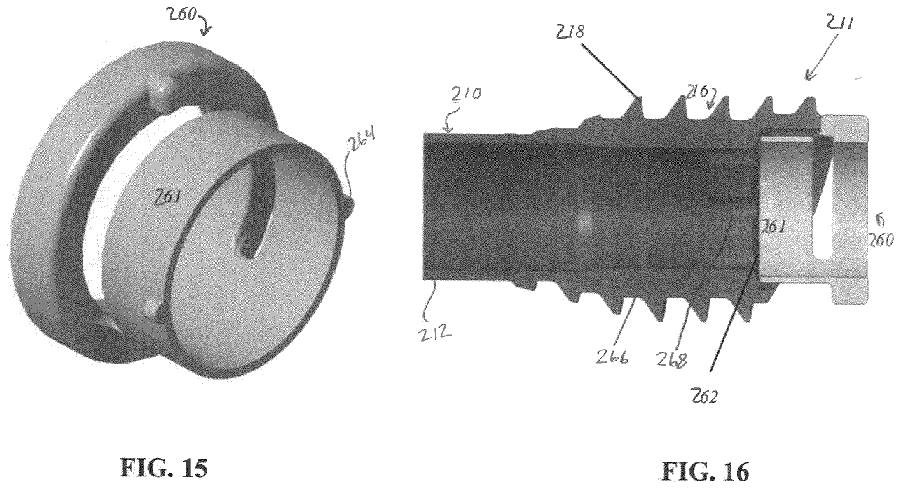

Each of FIGS. 15 and 16 illustrates a third headless head extension 260. The third headless head extension 260 is configured to be proximally loaded onto the bone screw 210. The third headless head extension 260 connects to the proximal portion 216 by using a first clip member 261 and a first clip member receiver 262. The third headless head extension 260 is positioned to extend the proximal portion 216, increasing the interface between the bone and the bone screw 210. The first clip member 261 is cylindrical and arched. However, the first clip member 261 can be any suitable shape. The arched shape of the clip member 261 is beneficial at least because it increases the stability of the connection by distributing forces more evenly across the proximal portion 216. Additionally, the first clip member 261 contains a plurality of notches 264 configured to interact with the first clip member receiver 262 located within a lumen 266 of the proximal portion 216. The plurality of notches 264 on the first clip member 261 aligns with corresponding notches 268 within the first clip member receiver 262. When sufficient linear force is applied, the clip member 261 and receiver 262 interlock, securing the third headless head extension 260 onto the proximal portion 216. The third headless head extension 260 is configured to abut the proximal portion 216 upon reaching the interlock point, with this abutment serving as a stopping point to ensure proper alignment and fitment.

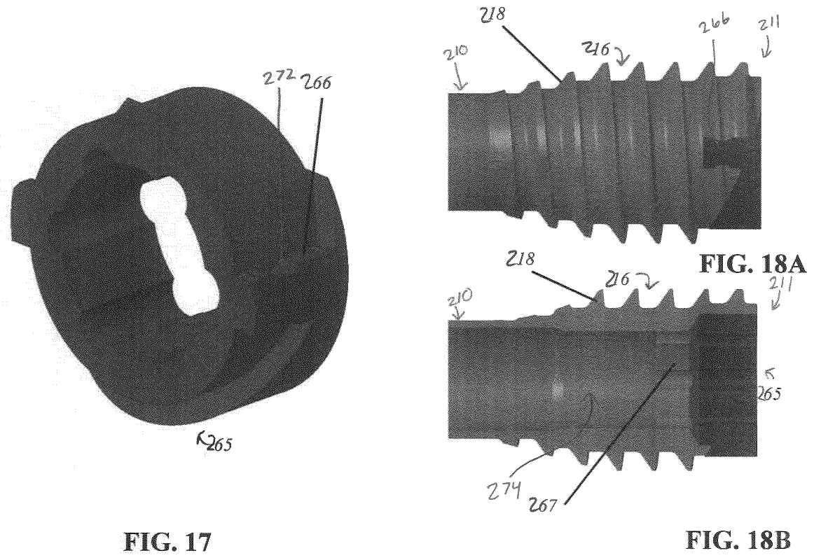

Each of FIGS. 17, 18A, and 18B illustrates a fourth headless head extension 265. The fourth headless head extension 265 is positioned to extend from the proximal portion 216. The fourth headless head extension 265 is configured to be preloaded into the bone screw 210 during assembly. The fourth headless head extension 265 interlocks with the proximal portion 216 to increase the interface between the bone and the bone screw 210.

The fourth headless head extension 265 further includes a second clip member 266. The second clip member 266 is designed with a plurality of notches 272 that are specifically configured to interact with the second clip member receiver 267, which is located within a lumen 274 of the proximal portion 216. The plurality of notches 272 on the second clip member 266 may allow for a secure interlock between the fourth headless head extension 265 and the proximal portion 216, ensuring proper alignment and preventing unwanted rotation or displacement of the fourth headless head extension 265 when in place. Additionally, if the second clip member 266 is broken away during surgical manipulation, the proximal portion 216 is beveled. The second clip member 266 and the second clip member receiver 267 are configured to separate in response to linear force.

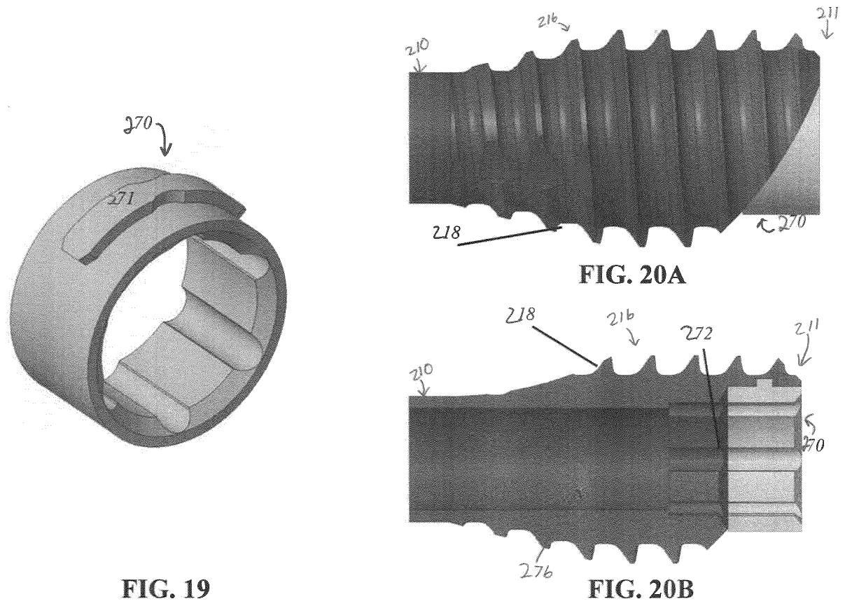

Each of FIGS. 19, 20A, 20B, and 21 illustrates a fifth headless head extension 270, which is configured to engage with the bone screw 210 through rotational movement. The fifth headless head extension 270 is positioned proximally onto the proximal portion 216. The fifth headless head extension 270 is loaded onto the bone screw 210 through a rotational action. The fifth headless head extension 270 includes a third clip member 271, which is cylindrical in shape. This cylindrical structure allows the third clip member 271 to be inserted into and rotate within the proximal portion 216. Additionally, the third clip member 271 is arched. The third clip member 271 may interact with the third clip member receiver 272 located within a lumen 276 of the bone screw 210. This interaction allows the fifth headless head extension 270 to interlock with the proximal portion 216 once the third clip member 271 is rotated into position. The third clip member receiver 272 is defined within the lumen 276 of the bone screw 210 and is specifically designed to engage the third clip member 271. When torque is applied to the fifth headless head extension 270, the third clip member 271 interlocks with the third clip member receiver 272.

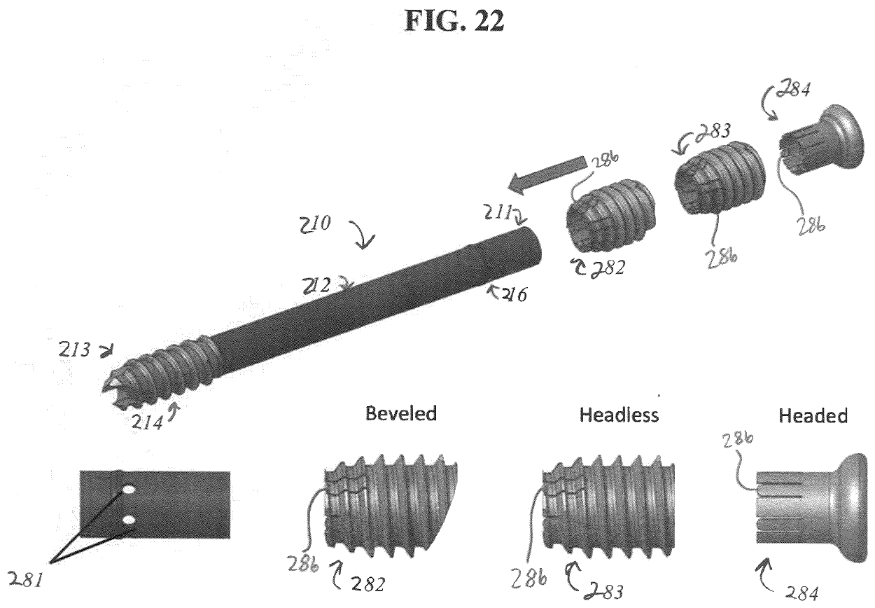

Each of FIGS. 22, 23, 24A, 24B, and 24C illustrates clip on head extension configurations. In these embodiments, the proximal portion 216 is configured to receive one of a clip on beveled, headless, or headed head extension (282, 283, 284). The proximal portion 216 is blank until merging with one of the clip on head extensions (282, 283, 284). The proximal portion 216 is configured to sit within the clip on head extension (282, 283, 284). Even further, in these embodiments, the proximal portion 216 includes a plurality of apertures 281. The plurality of apertures 281 are configured to receive clip members 286 from a desired clip on head extension (282, 283, 284).

Those with ordinary skill in the art will appreciate that various modifications and alternatives for the described and illustrated examples can be developed in light of the overall teachings of the disclosure, and that the various elements and features of one example described and illustrated herein can be combined with various elements and features of another example without departing from the scope of the invention. Accordingly, the particular arrangements of elements and steps disclosed herein have been selected by the inventor simply to describe and illustrate examples of the invention and are not intended to limit the scope of the invention or its protection, which is to be given the full breadth of the appended claims and any and all equivalents thereof.

What is claimed is:

1. A bone screw comprising:
a body having a body proximal end, a body distal end, and a body length extending between the body proximal end and the body distal end;
a proximal portion having a proximal portion proximal end, a proximal portion external thread, and a proximal portion length extending from the proximal portion proximal end to the body proximal end, the proximal portion length being less than the body length;
a distal tip having a cutting surface and extending from the body distal end; and
a head extension disposed on the proximal portion and having a head extension internal thread threaded with the proximal portion external thread form.

2. The bone screw of claim 1, wherein the head extension has a head extension proximal end;
wherein the head extension and the proximal portion taper from the head extension proximal end to the body proximal end.

3. The bone screw of claim 1, wherein the head extension proximal end has an interrupted dome.

4. The bone screw of claim 1, wherein the proximal portion proximal end is beveled.

5. The bone screw of claim 1, wherein the head extension has a head extension distal end; and
wherein the head extension distal end is beveled.

6. The bone screw of claim 1, wherein the proximal portion has a proximal portion external diameter;
wherein the body has a body external diameter;
wherein the distal tip has a distal tip external diameter; and
wherein the proximal portion external diameter is greater than the body external diameter and the distal tip external diameter.

7. The bone screw of claim 1, wherein the body and the distal tip are a uniform piece.

8. The bone screw of claim 1, wherein the head extension defines a head extension inner diameter;
wherein the proximal portion defines a proximal portion outer diameter; and
wherein the head extension inner diameter is greater than the proximal portion outer diameter.

9. The bone screw of claim 8, wherein the distal tip defines a distal tip outer diameter; and
wherein the head extension inner diameter is greater than the distal tip outer diameter.

10. The bone screw of claim 9, wherein the body defines a body outer diameter; and
wherein the head extension inner diameter is greater than the body outer diameter.

11. The bone screw of claim 1, wherein the head extension includes a head extension external thread; and
wherein the head extension external thread and the proximal portion external thread form are continuous.

12. The bone screw of claim 1, wherein the proximal portion is tapered from the proximal portion proximal end to the body proximal end.

13. The bone screw of claim 1, wherein the head extension abuts the proximal portion.

14. The bone screw of claim 1, wherein the head extension is a separate component from the proximal portion.

15. The bone screw of claim 14, wherein the head extension is configured to be loaded proximally onto the body.

16. The bone screw of claim 1, wherein the head extension and the proximal portion are made from the same material.

17. The bone screw of claim 1, wherein the head extension and the proximal portion are made from different materials.

18. The bone screw of claim 1, wherein the head extension is an interrupted dome.

19. A bone screw comprising:
a body having a body proximal end, a body distal end, and a body length extending between the body proximal end and the body distal end;
a proximal portion having a proximal portion proximal end, a proximal portion external thread form, and a proximal portion length extending from the proximal portion proximal end to the body proximal end, the proximal portion length being less than the body length;
a distal tip having a cutting surface and extending from the body distal end; and
a head extension disposed on the proximal portion and having a head extension proximal end and a head extension internal thread threaded with the proximal portion external thread form;
wherein the head extension and the proximal portion taper from the head extension proximal end to the body proximal end; and
wherein the proximal portion proximal end is beveled.

20. A bone screw comprising:
a body having a body proximal end, a body distal end, and a body length extending between the body proximal end and the body distal end;
a proximal portion having a proximal portion proximal end, a proximal portion external thread form, and a proximal portion length extending from the proximal portion proximal end to the body proximal end, the proximal portion length being less than the body length;
a distal tip having a cutting surface and extending from the body distal end; and
a head extension disposed on the proximal portion and having a head extension proximal end, a head extension external thread, and a head extension internal thread threaded with the proximal portion external thread form;
wherein the head extension and the proximal portion taper from the head extension proximal end to the body proximal end;
wherein the proximal portion proximal end is beveled; and
wherein the head extension thread and the proximal portion external thread form are continuous.
